(19) United States
(12) Patent Application Publication
(10) Pub. No.: US 2009/0196802 A1
(43) Pub. Date: Aug. 6, 2009

(54) DISINFECTION SYSTEM WITH AN IMMERSION EMITTER

(75) Inventor: Hans-Jürgen Streppel, Wipperfürth (DE)

Correspondence Address: OSRAM Sylvania Inc.;Attn. Ms. Sandi Basti, 100 Endicott Street, Danvers, MA, 01923, US

(73) Assignee: Osram Gesellschaft Mit Beschraenkter Haftung, Munich (DE)

(21) Appl. No.: 11/813,372

(22) PCT Filed: Dec. 20, 2005
(86) PCT No.: PCT/DE05/02296

§ 371 Date: Jul. 5, 2007

(30) Foreign Application Priority Data
Jan. 5, 2005 (DE) 10 2005 000 845.3

(51) Int. Cl. A61L 2/10; B01J 19/12; C02F 1/32

(52) U.S. Cl. 422/186.3

(57) ABSTRACT

A disinfection system that is used to disinfect liquids (4) in a container (6), includes an immersion emitter (8) which is immersed at least partially in the liquid that is to be disinfected via an opening in the container (6) and which emits UV rays. According to the invention, the disinfection system (2) is provided with a safety cover (10) which is used seal the opening, the immersion emitter (8) being suspended thereon.

### TECHNICAL FIELD

The invention relates to a disinfection system for disinfecting liquids in accordance with the preamble of patent claim 1.

### PRIOR ART

In order to avoid diseases, drinking water needs to satisfy certain quality standards. Owing to incorrect storage, contaminated filling and removal stations, however, bacteria, viruses and other pathogens can collect in the drinking water which make it undrinkable. Purifying such contaminated drinking water takes place in known disinfection systems via the addition of a chemical substance such as chlorine, silver ions, for example, or by a mechanical filter. One disadvantage associated with the addition of a chemical substance, however, is the fact that predetermined doses need to be precisely adhered to in order to avoid any risk to humans and the environment. Disadvantages associated with the mechanical filter are the fact that the disinfective effect is reduced over time and the filter is quite complex to clean.

Other alternative disinfection systems envisage subjecting the drinking water to ultraviolet radiation (UV-C radiation). Such a disinfection system is disclosed, for example, in U.S. Pat. No. 6,042,720, in which the drinking water is treated in a special disinfection container and is purified in this container by means of UV irradiation. One disadvantage with this solution is the fact that it is not possible to rule out the purified drinking water being recontaminated once it has been removed from the disinfection container and has been refilled into a storage container. In this case, the recontamination can result in particular from dirt located in the storage container.

A disinfection system for purifying the drinking water in a storage container is described in U.S. Pat. No. 5,900,212. The disinfection system has a UV immersion emitter, a section of which is immersed in the drinking water in the storage container and, in the process, the emitter is supported on an opening wall and on the base of the container. This solution has the disadvantage that the storage container is open during disinfection, with the result that further contamination is not ruled out. Furthermore, this solution has the disadvantage that the position of the immersion emitter in the storage container cannot be fixed precisely, with the result that defined irradiation is not possible and there is therefore the risk of the drinking water not being sufficiently disinfected.

### DESCRIPTION OF THE INVENTION

The invention is based on the object of providing a low-maintenance disinfection system for disinfecting liquids in a container, which allows for reliable and efficient disinfection and is cost-effective in terms of production and in operation.

This object is achieved according to the invention by a disinfection system for disinfecting liquids in a container with an immersion emitter, at least a section of which is immersed in the liquid to be disinfected through an opening of the container and which emits UV rays, characterized in that the disinfection system has a safety cover for closing the opening, on which the immersion emitter is suspended. Particularly advantageous embodiments of the invention are described in the dependent claims.

The disinfection system according to the invention for disinfecting liquids in a container has an immersion emitter, at least a section of which is immersed in the liquid to be disinfected through an opening of the container and which emits UV rays. According to the invention, the disinfection system has a safety cover for closing the opening, on which the immersion emitter is suspended. One advantage of this solution is the fact that the disinfection can take place in any desired container and not in a special disinfection container, with the result that reliable disinfection is possible since, in addition to the liquid, also the container is purified with each UV treatment. Further advantages are the fact that the container is sealed during purification and that the immersion emitter assumes a defined position in the container or the liquid, with the result that efficient UV treatment can take place.

In a preferred embodiment, the immersion emitter is suspended on the safety cover via its power supply cable, with the result that additional suspension means can be dispensed with. In order to open the container quickly, the safety cover interacts with a mounting flange, which is connected detachably to the container in the region of the opening and does not need to be removed in order to remove the safety cover.

Ideally, the bearing length of the power supply cable can be varied by means of a fixing device on the safety cover, with the result that the depth of immersion of the immersion emitter can be adjusted.

The power supply cable can be split in two, the immersion emitter being suspended on a first cable section, and a switched mode power supply or a battery for supplying power being connected to a second cable section. The electrical connection between the two cable sections can be interrupted. Preferably, for interruption purposes a switch is provided which interrupts the power supply to the immersion emitter when the safety cover is removed.

In order to deliver the purified liquid from the container, leadthroughs for a delivery line and a supply line of a pump which can be arranged in the container can be provided in the safety cover and the mounting flange.

The immersion emitter can be made heavier by an additional weight in order for it to automatically be immersed in the liquid.

The immersion emitter may have a UV lamp, which is accommodated in a quartz glass tube. The quartz glass tube is sealed at the ends via two terminating pieces, at least sections of which can be inserted into the quartz glass tube. Advantageously, the weight is integrated in one of the terminating pieces.

### BRIEF DESCRIPTION OF THE DRAWINGS

The invention will be explained in more detail below with reference to preferred exemplary embodiments. In the drawings:

FIG. 1 shows an installed disinfection system according to the invention,

FIG. 2 shows a detailed overall view of the exemplary embodiment from FIG. 1,

FIG. 3 shows a longitudinal section through the lower terminating piece from FIG. 2,

FIG. 4 shows a longitudinal section through the upper terminating piece from FIG. 2,

FIG. 5 shows a view of the safety cover from FIG. 2, from below,

FIG. 6 shows a plan view of the mounting flange from FIG. 2, and

FIG. 7 shows a longitudinal section through the mounting flange from FIG. 2.

### PREFERRED EMBODIMENT OF THE INVENTION

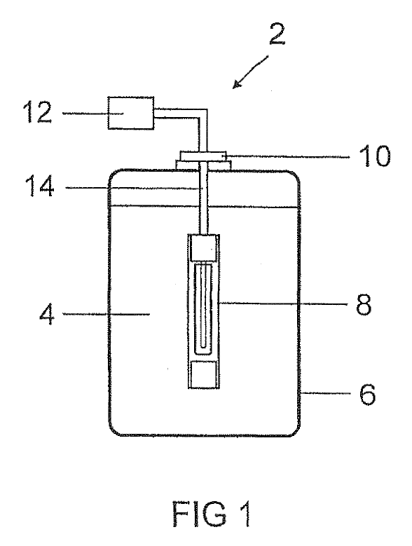

FIG. 1 shows a disinfection system 2 according to the invention for disinfecting liquids 4 in a container 6. The container 6 is a conventional storage container having a standard closure for storing drinking water, for example, such as is widely used in the leisure camping or expedition sectors. The disinfection system 2 has an immersion emitter 8, a safety cover 10 and a switched mode power supply 12. The immersion emitter 8 is immersed in the liquid 4 through an opening (not illustrated) of the container 6 and emits UV radiation, more precisely UV-C radiation. The safety cover 10 closes the opening, the immersion emitter 8 being connected to the switched mode power supply 12 for supplying power via a power supply cable 14, which at the same time acts as a suspension means.

The disinfection system 2 is simple to install and allows reliable and efficient disinfection. Preferably, the UV-C radiation has a wavelength of 253.7 nm and kills off the germs found in the liquid 4. The immersion emitter 8 preferably has a power of 9 W and is suitable for a 10 l to 20 l container 6 without any additional circulation at exposure times of approximately 5 min. Larger volumes can be reliably disinfected by correspondingly lengthening the exposure time and/or circulation. In order to prevent the UV-C radiation emerging from the container 6, the container is designed to be UV-absorbent. In order to irradiate the liquid 4 uniformly, the immersion emitter 8 shown in the figure is positioned in the center of the container 6. The maintenance of the immersion emitter 8 is substantially restricted to regular removal of the dirt deposited on the immersion emitter 8.

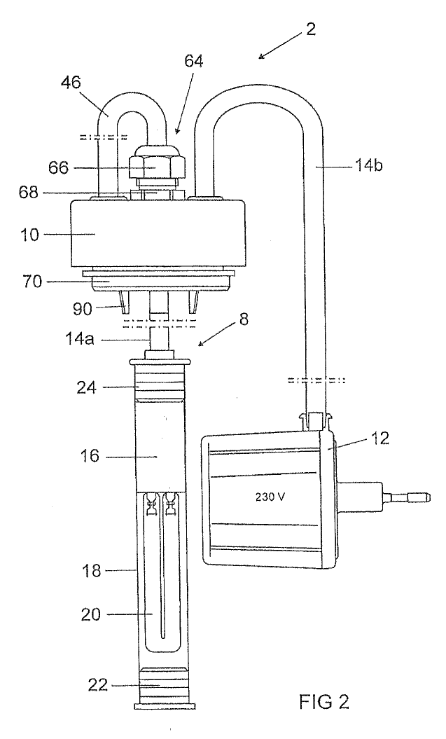

FIG. 2 shows the disinfection system 2 from FIG. 1 in a detailed overall view. The immersion emitter 8 has a quartz glass tube 18, in which a conventional UV lamp 16 with a U-shaped lamp vessel 20 is accommodated. The quartz glass tube 18 is closed in a sealing manner at the ends by a lower and an upper terminating piece 22, 24 as shown in FIG. 2, at least sections of which terminating piece are inserted.

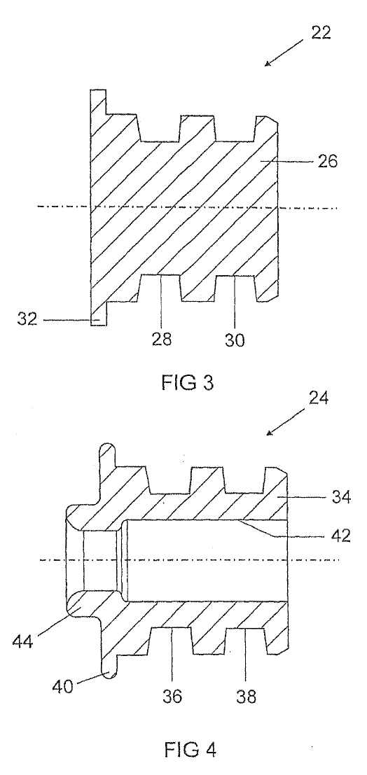

As shown in the longitudinal section in FIG. 3, the lower terminating piece 22 has a cylindrical basic body 26, in which two circumferential grooves 28, 30, are introduced for the purpose of accommodating UV-C-resistant O sealing rings. In addition, the lower terminating piece 22 has a radially extended annular face 32, with which it bears on the end side against the quartz glass tube 18, in the inserted state. The lower terminating piece 22 is designed in terms of weight in such a way that the immersion emitter 8 is made heavier and is automatically immersed in the liquid 4. It is possible to envisage, for example, manufacturing the terminating piece 26 from stainless steel.

As shown in the longitudinal section in FIG. 4, the upper terminating piece 24 likewise has a cylindrical basic body 34, in which two circumferential grooves 36, 38 are formed for the purpose of accommodating UV-C-resistant O sealing rings. In addition, it has a radially extended annular face 40 for delimiting the insertion depth into the quartz glass tube 18. Additionally, the upper terminating piece 24 has an axial drilled hole 42 passing through it for the purpose of leading through the power supply cable 14. The terminating piece 24 has, on its side remote from the annular face 40, an axial projection 44 in a form of an anti-kink means for the power supply cable 14. In order to prevent the ingress of the liquid 4 into the quartz glass tube 18 along the power supply cable 14, the axial drilled hole 42 is correspondingly radially tapered in the region of the projection 44, with the result that the power supply cable 14 bears in a sealing manner against the inner circumferential wall of the axial drilled hole 42.

The power supply cable 14 is split in two via two cable sections 14a, 14b (FIG. 2). The first cable section 14a is connected to the immersion emitter 8, and the second is connected to the switched mode power supply 12. The switched mode power supply 12 may be, for example, a 230 V transformer having an electronic (EB) or conventional (CB) ballast or a 12 V battery having such a ballast. The exemplary embodiment with the 12 V battery provides the advantage that the immersion emitter 8 can be operated using a car battery. Both cable sections 14a, 14b can be electrically connected to one another in the safety cover 10 such that the lamp 16 of the immersion emitter 8 can emit UV-C radiation. The first cable section 14a passes through the safety cover 10, its free end section being passed back into the safety cover 10, with the result that a cable loop 46 is formed, via which the depth of immersion of the immersion emitter 8 can be varied.

FIG. 5 shows a view of the safety cover 10 from FIG. 2, from below. The safety cover 10 is an injection-molded part and has a central drilled hole 48 for leading through the first cable section 14a and a drilled through-hole 50 for accommodating the free end section of the first cable section 14a. Furthermore, a drilled through-hole 52 for accommodating the free end section of the second cable section 14b is provided. The cable sections 14a, 14b each open out into a kidney-shaped cutout 54, 56. In the right-hand cutout 54 in FIG. 5, a cable clamp 58 for connecting the first cable section 14a is provided. In the left-hand cutout 56 in FIG. 5, a switch (not illustrated), preferably a magnetic switch, is fixed to two pins 57, 59 and connected to the second cable section 14b. In order to secure the switch, it is cast in the right-hand cutout 56. The cable clamp 58 and the switch are electrically connected to one another.

In addition, the safety cover 10 has two prepunched round surface sections 60, 62 for leading through a delivery line (not illustrated) and an electrical supply line for a pump, preferably a hose pump, which can be arranged in the container 6, for delivering the liquid 4. In order to be able to use pumps with different delivery powers, the surface sections 60, 62 can be punched with different diameters.

As shown in FIG. 2, a fixing device 64 is provided on the safety cover 10 for the purpose of adjusting the led-through length of the first cable section 14a or the depth of immersion of the immersion emitter 8 into the liquid 4 in the region of the central drilled hole 48. It essentially has a nut 66, which is operatively connected to a threaded connection piece 68, which engages in a clamping manner around the first cable section 14a when the nut 66 is tightened.

The safety cover 10 is connected to the container 6 via a mounting flange 70 surrounding the opening. The mounting flange 70 is likewise an injection-molded part and is designed such that it can be used with known closure systems such as screw-type closures according to DIN 96 with an opening diameter of 100 mm or 120 mm, for example.

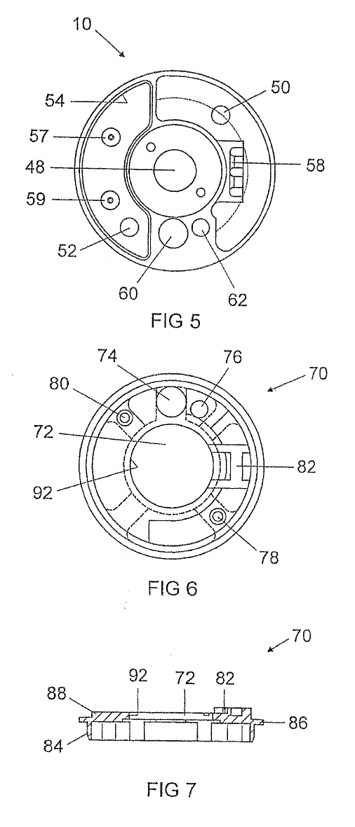

As shown in the plan view in FIG. 6, the mounting flange 70 has an axial drilled hole 72 for the purpose of leading through the immersion emitter 8 and two drilled holes 74, 76 for the purpose of leading through the delivery line and the supply line for the pump. In order to be able to fix the safety cover 10 also to containers which do not have a screw-type closure, two diametric drilled through-holes 78, 80 are provided for screws which can be screwed into corresponding threaded holes of the container.

For safety reasons, it is advantageous if the electrical connection between the two cable sections 14a, 14b and therefore the power supply to the immersion emitter 8 is interrupted when the safety cover 10 is lifted off and only closed again when the safety cover 10 is positioned again. This is realized in the present embodiment by virtue of the fact that the mounting flange 70 has a holder 82 for accommodating a magnet (not illustrated) of the magnetic switch, with the result that the switching position of the switch can be varied by means of the distance between the safety cover 10 and the mounting flange 58.

It can be seen in the sectional illustration in FIG. 7 that the mounting flange 70, for the purpose of guiding it on the container 6, has an axial guide projection 84, which extends into the container opening, and a fixing shoulder 86 to be fixed to said guide projection, with which fixing shoulder 86 it is supported on the edge of the opening, on the one hand, and, on the other hand, which fixing shoulder 86 can be surrounded by a rotary ring (not illustrated) of the screw-type closure, with the result that the mounting flange 70 is pressed against the edge of the opening and is fixed detachably to the container 6. In addition, the mounting flange 70 has a cylindrical body section 88, a section of which, in the joined state, are immersed in the safety cover 10 and bear against the inner circumference thereof.

As shown in FIG. 2, the safety cover 10 is fixed to the mounting flange 70 via a large number of axially extending tongues 90. These tongues 90 are fixed to the safety cover 10 and designed such that they are immersed in the axial drilled hole 72 in the mounted state and interact in an interlocking manner with the circumferential wall 92 of the axial drilled hole 72. When the safety cover 10 is positioned onto the mounting flange 70 mounted on the container 6, the tongues 90 latch with the circumferential wall 92, with the result that the safety cover 10 is safely connected to the mounting flange 70 and therefore to the container 4. In order to detach this connection, the safety cover 10 needs to be moved in such a way that the tongues are brought out of their interlocking engagement with the circumferential wall 92, the power supply to the immersion emitter 8 being interrupted by the increasing distance between the magnet and the magnetic switch.

The invention discloses a disinfection system for disinfecting liquids in a container with an immersion emitter, at least a section of which is immersed in the liquid to be disinfected through an opening of the container and which emits UV rays. According to the invention, the disinfection system has a safety cover for closing the opening, on which the immersion emitter is suspended.

1-11. (canceled)

12. A disinfection system for disinfecting liquids (4) in a container (6) with an immersion emitter (8), at least a section of which is immersed in the liquid (4) to be disinfected through an opening of the container (6) and which emits UV rays, characterized in that the disinfection system (2) has a safety cover (10) for closing the opening, the immersion emitter (8) being suspended on the safety cover (10) via a power supply cable (14).

13. The disinfection system as claimed in claim 12, the safety cover (10) being connected to a mounting flange (70), which is fixed detachably to a container section in the region of the opening.

14. The disinfection system as claimed in claim 12, it being possible to adjust the depth of immersion of the immersion emitter (8) via the bearing length of its power supply cable (14).

15. The disinfection system as claimed in claim 13, it being possible to adjust the depth of immersion of the immersion emitter (8) via the bearing length of its power supply cable (14).

16. The disinfection system as claimed in claim 14, the safety cover (10) having a fixing device (64) for clamping the power supply cable (14).

17. The disinfection system as claimed in claim 16, the power supply cable (14) being split in two, and the immersion emitter (8) being suspended on a first cable section (14a), and a second cable section (14b) being connected to a switched mode power supply (12) or a battery, an electrical connection between the two cable sections (14a, 14b) being capable of being interrupted.

18. The disinfection system as claimed in claim 17, the electrical connection between the two cable sections (14a, 14b) being capable of being interrupted by the safety cover (10) being removed from a mounting flange (70).

19. The disinfection system as claimed in claim 13, leadthroughs (60, 62, 74, 76) for accommodating a delivery line and a supply line for a pump in the container (6) for delivering the liquid (4) being formed in the safety cover (10) and the mounting flange (70).

20. The disinfection system as claimed in claim 12, the immersion emitter (8) being provided with an additional weight.

21. The disinfection system as claimed in claim 12, a lamp vessel (20) for producing the UV radiation being accommodated in a quartz glass tube (18), which is sealed by means of two terminating pieces (22, 24), sections of which are inserted at one end into the quartz glass tube (18).

22. The disinfection system as claimed in claim 20, the additional weight being integrated in a terminating piece (22).
